(19) United States
(12) Patent Application Publication
(10) Pub. No.: US 2017/0348000 A1
(43) Pub. Date: Dec. 7, 2017

(54) SURGICAL FIXATION ASSEMBLIES, SYSTEMS AND KITS

(71) Applicant: THE CLEVELAND CLINIC FOUNDATION, Cleveland, OH (US)

(72) Inventors: Noble G. Jones, Cleveland Hts., OH (US); Matthew Kroh, Cleveland, OH (US)

(21) Appl. No.: 15/615,267

(22) Filed: Jun. 6, 2017

Related U.S. Application Data
(60) Provisional application No. 62/346,019, filed on Jun. 6, 2016.

(51) Int. Cl. A61B 17/064; A61B 17/068

(52) U.S. Cl. CPC A61B 17/0643; A61B 17/068

(57) ABSTRACT

Surgical stapling devices including surgical fastening assemblies, surgical fastening devices, surgical fastening kits, and surgical fastening systems are provided. Surgical fastening assemblies include staples that provide a redundant approach and have prongs that are retroflexed onto the back of opposing staples or provide a rivet based compression. Surgical fastening devices include low profile fastening devices that can be attached to the distal end of endoscopes. Surgical stapling systems include combinations of staples, rivets, fastening devices and/or endoscopes to which fastening devices are attached.

### RELATED APPLICATIONS

This application claims priority from U.S. Patent Application Ser. No. 62/346,019, filed 6 Jun. 2016, which is incorporated herein in its entirety.

### TECHNICAL FIELD

The present disclosure generally relates to surgical staple assemblies, surgical rivet assemblies, surgical fastening devices, surgical fastening systems, and surgical fastening kits.

### BACKGROUND

Gastrointestinal (GI) perforations can form along the GI tract due to a number of causes such as, for example, diverticulitis, appendicitis, peptic ulcers, non-steroidal anti-inflammatory drugs (NSAIDS), steroids, and trauma. The decision to perform corrective surgery is contingent upon a thorough assessment of the benefits versus the risks associated with post-operative sequelae. Current surgically corrective actions primarily include the use of open and laparoscopic approaches. Although laparoscopic surgery is minimally invasive and substantially reduces the risk of surgical complications when compared to some open procedures, the use of trocars to provide access to the abdominal cavity still presents surgical risks. Risks include the perforation of healthy organs upon percutaneous entry into the abdomen and wound infection. Current flexible endoscopic based therapies include the use of endoscopic clip delivery systems which has significant perforation size limitations and a suturing system that is effective, but requires extensive training even for an experienced endoscopist to become a proficient operator.

There is a need for surgical fastening devices and systems that minimize or eliminate the above-mentioned risks and complications by not requiring percutaneous entry to make gastrointestinal perforation repairs, while still offering ease of use for an endoscopist.

The present disclosure generally provides surgical staples, surgical staple assemblies, surgical fastening devices, surgical fastening systems, surgical rivet assemblies, and surgical fastening kits. An embodiment provides a surgical duplex staple assembly comprising a first staple and a second staple. The first staple comprises a base having a back surface and a front surface. The base has a top portion, a bottom portion, a left side portion, and a right side portion. The first staple further includes a left prong extending from the left side portion and a right prong extending from the right side portion. The second staple comprises a base having a back surface and a front surface. The second staple has a top portion, a bottom portion, a left side portion and a right side portion. The second staple further includes a top prong extending from the top portion and a bottom prong extending from the bottom portion. In a deployed position, the left and right prongs of the first staple penetrate through the approximated tissue and are retroflexed onto the back surface of the second staple. Further, the top and bottom prongs of the second staple penetrate through the approximated tissue and are retroflexed onto the back surface of the first staple. Other embodiments of a staple include a 4-pronged first staple and a second staple backing without prongs. In a deployed position, the prongs of the first staple penetrate through the approximated tissue and retroflex onto the back surface of the second staple backing. The first or second staple can include different numbers of prongs.

Other embodiments provide surgical fastening devices that can be used to deploy the duplex staple assembly described above, rivet assemblies as disclosed herein, or other fastening devices and other fasteners. In an embodiment, a fastening device comprises a casing have a first and a second carrier disposed therein that are connected by a carrier compression spring. The first carrier is configured to house a plurality of first staples or rivets of a rivet assembly. The first carrier has a distal end defining an exit opening through which the plurality of first staples or rivets can be sequentially deployed. The second carrier is configured to house a plurality of second staples or secondary heads of rivet assemblies therein. Each of the plurality of second staples is configured to fasten to a respective one of the plurality of first staples. Similarly, each of the secondary heads is configured to fasten to a respective one of the plurality of rivets. Similar to the first carrier, the second carrier has a distal end defining an exit opening through which the plurality of second staples or secondary heads can be sequentially deployed. The fastening device further comprises a first plunger disposed within the first carrier that actuates deployment of the plurality of first staples or rivets. The fastening device also includes a second plunger disposed within the second carrier that actuates deployment of the plurality of second staples or secondary heads.

In another embodiment, a fastening device comprises a clamp comprising opposing first and second arms. The first arm has an exit port at a distal end thereof through which a staple or rivet of a rivet assembly is deployed. The second arm has an exit port at a distal end thereof through which a complementary staple or secondary head of a rivet assembly is deployed. The fastening device further includes a spring-loaded casing in operable association with the clamp and located between the first and second arms of the clamp in a stowed position. The spring-loaded casing has a first section configured to house a plurality of first staples or rivets and a second section configured to house a plurality of complementary second staples or secondary heads. The first section has a distal access port facing the distal end of the first arm through which the first arm can capture a first staple or rivets of the plurality of first staples or rivets. Similarly, the second section has a distal access port facing the distal end of the second arm through which the second arm can capture a complementary second staple or secondary head of the plurality of complementary second staples or secondary heads. The fastening device also includes a rod in slidable relation with the clamp and located distal to the spring-loaded casing.

Other embodiments of the present disclosure provide surgical fastening systems and surgical fastening kits that include suitable combinations of the above-described staples, rivet assemblies, fastening devices, and endoscopes to which fastening devices are attached.

### BRIEF DESCRIPTION OF THE DRAWINGS

FIG. 1 is perspective view of a duplex staple assembly in a non-deployed position according to an embodiment of the present disclosure.

FIG. 2 is a perspective view of the duplex staple assembly of FIG. 1 in a deployed position with tissue not depicted for the purpose of clarity.

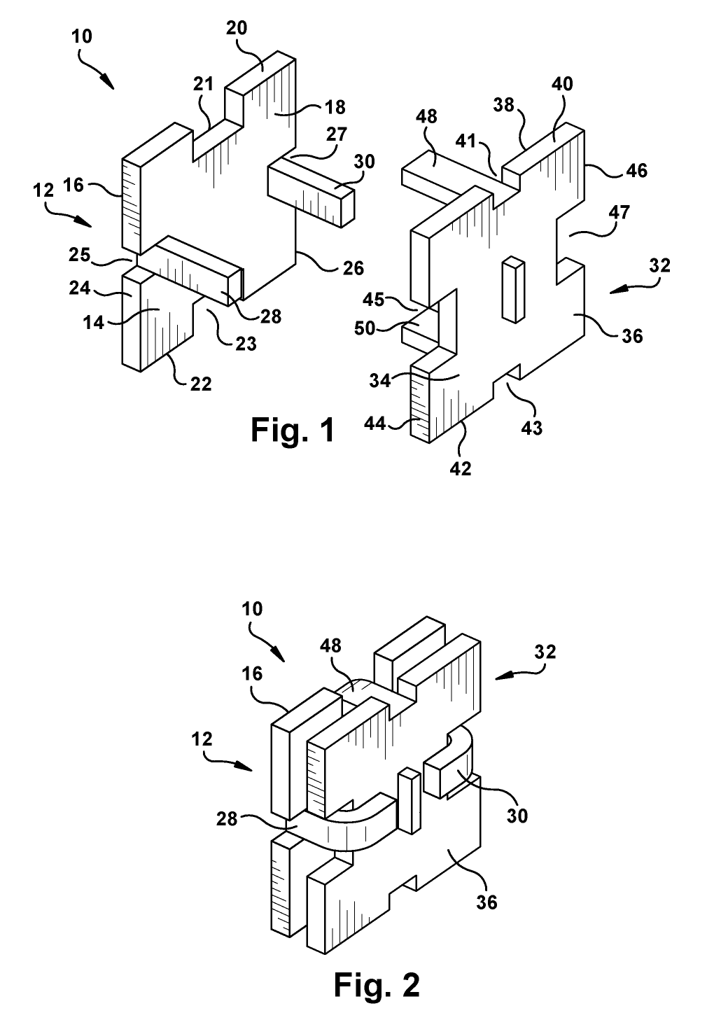

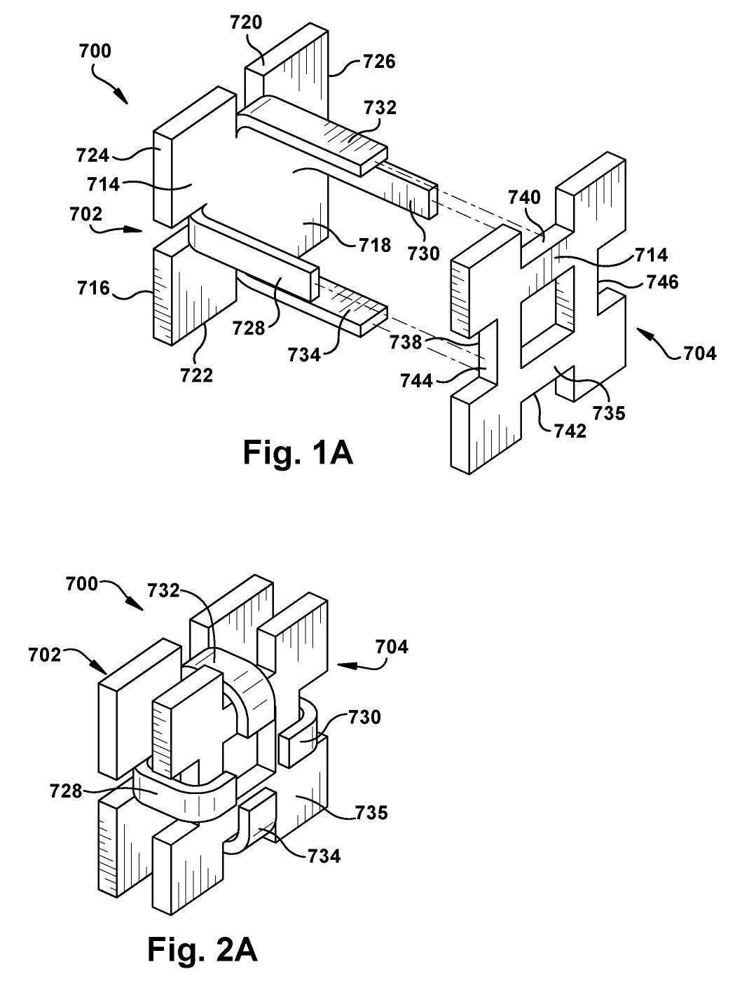

FIG. 1A is perspective view of a duplex staple assembly in a non-deployed position according to an embodiment of the present disclosure.

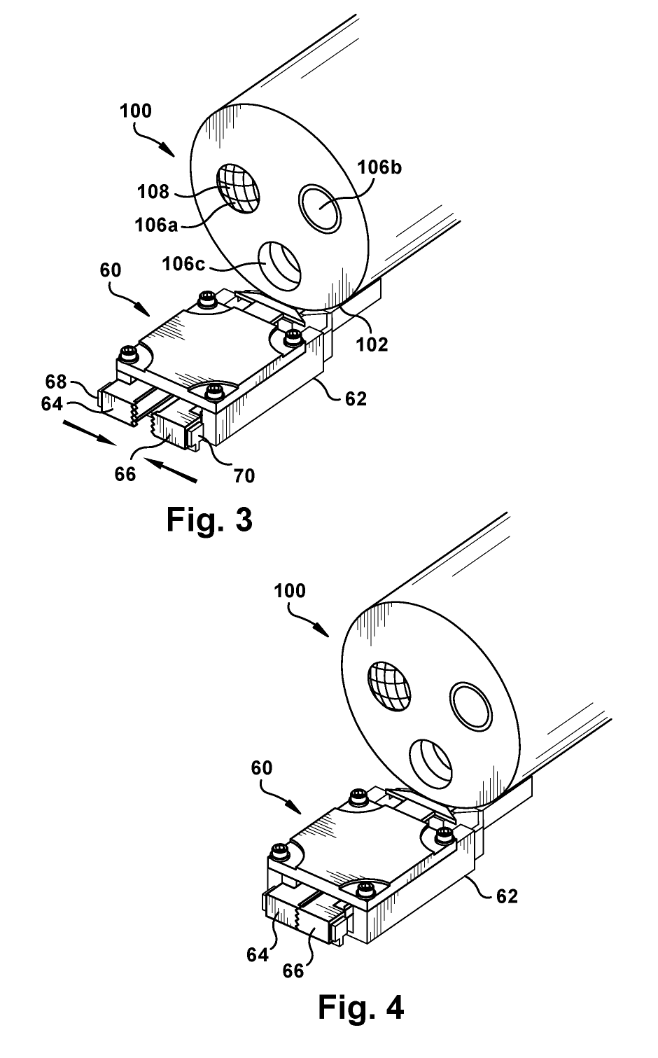

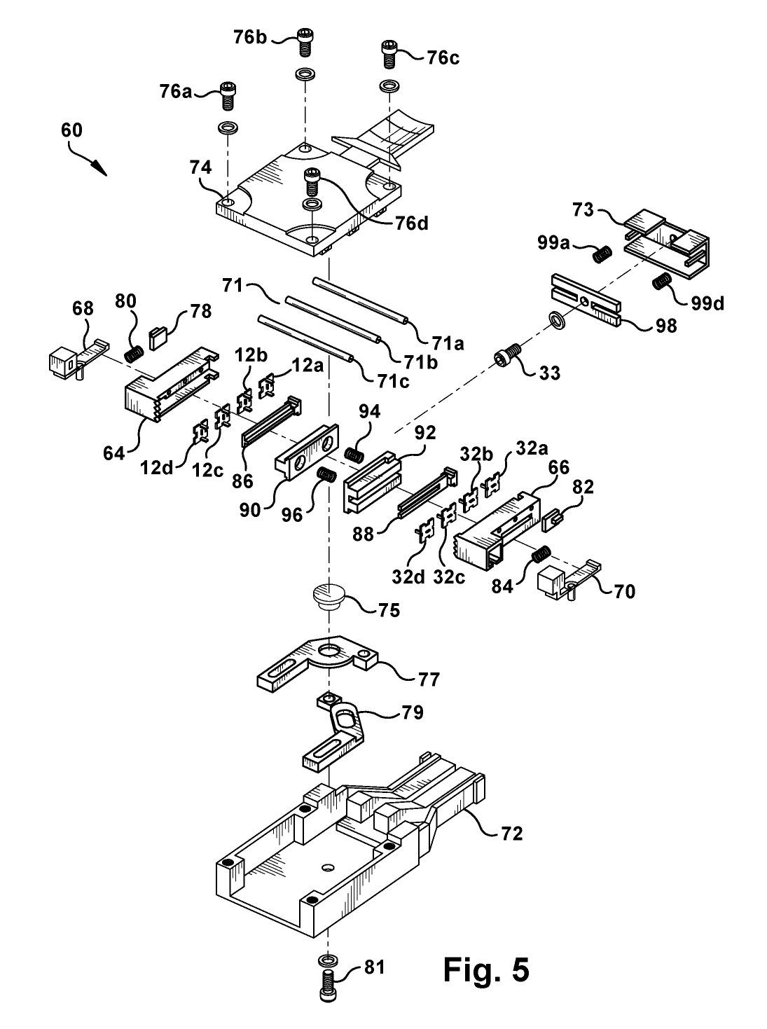

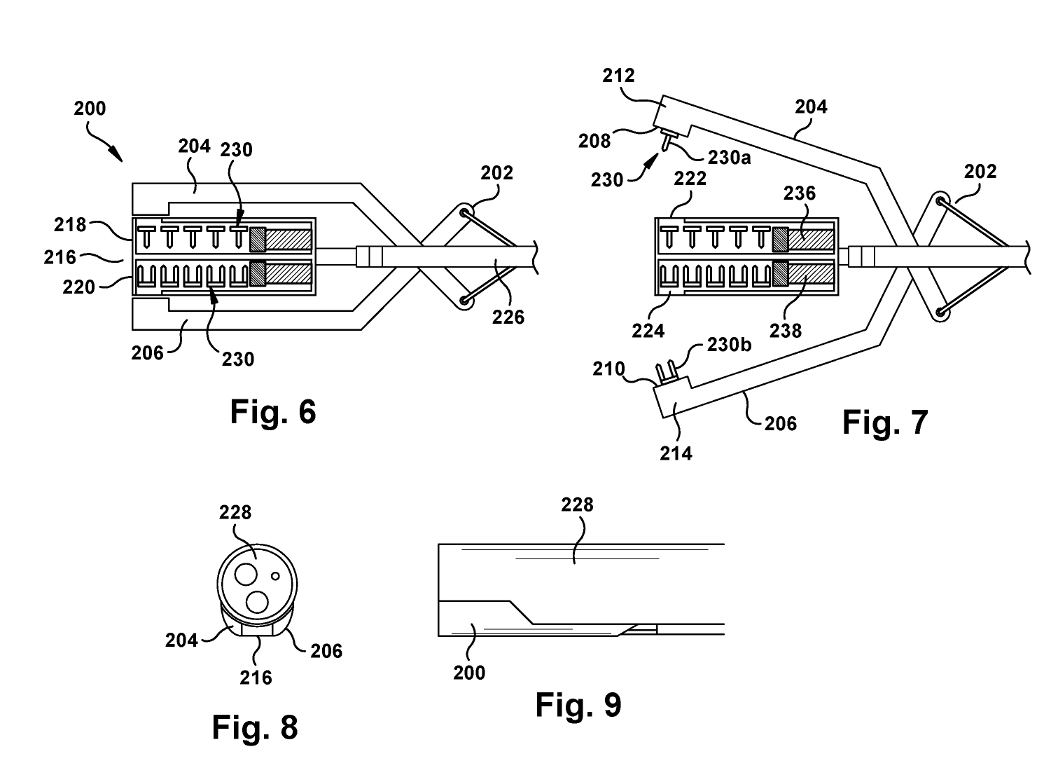

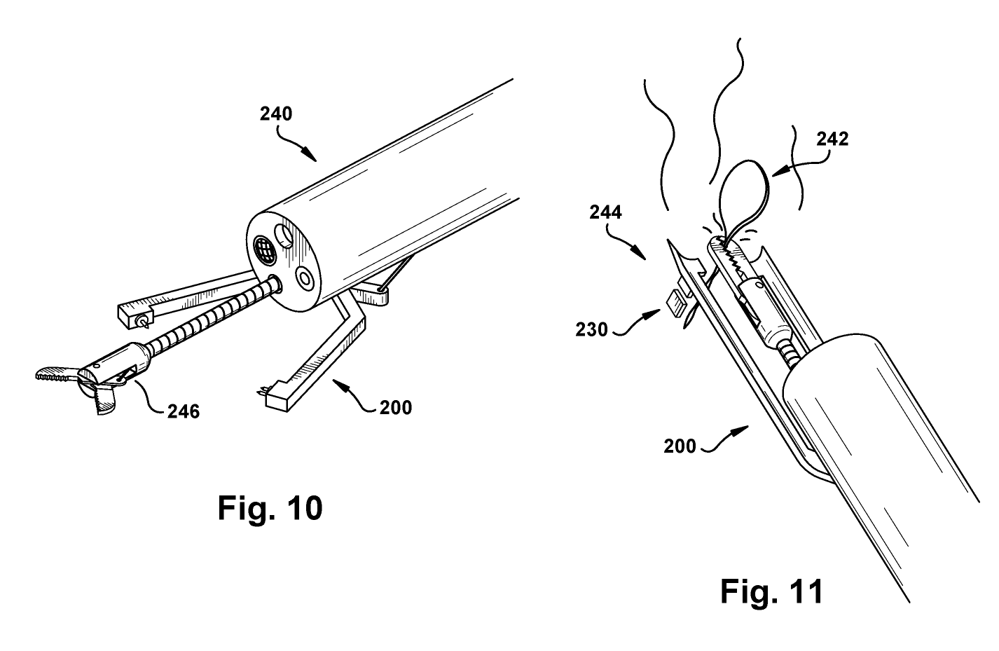

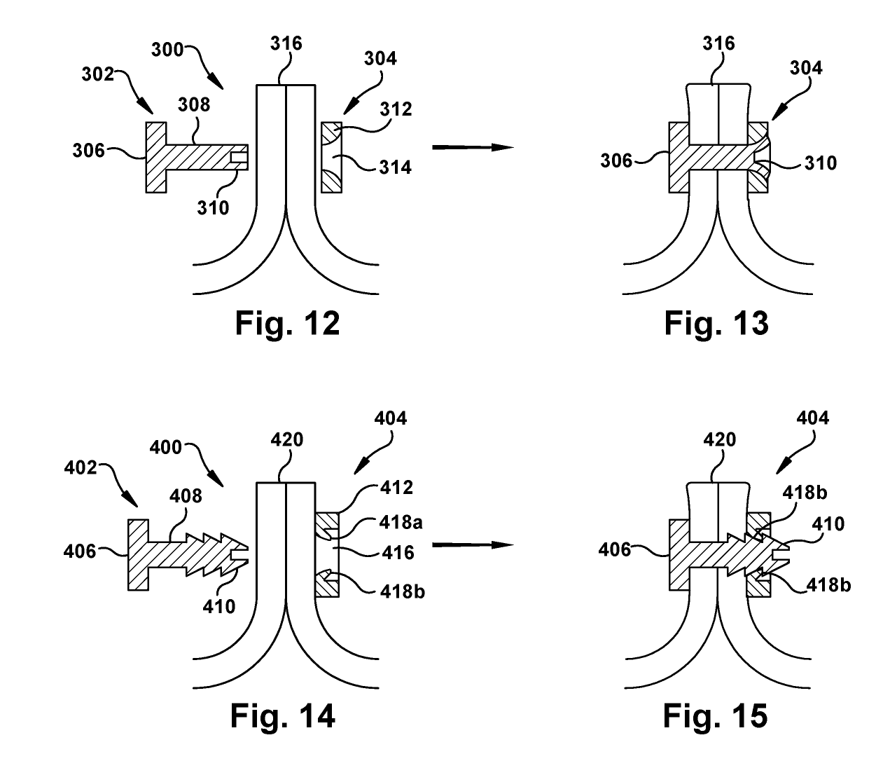

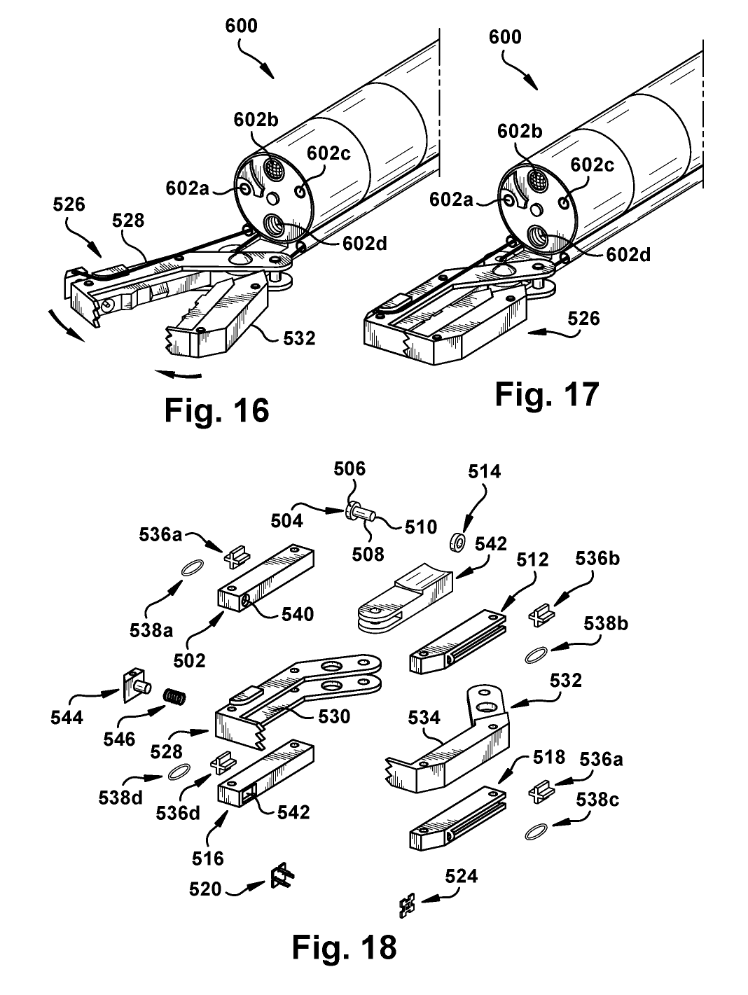

FIG. 2A is a perspective view of the duplex staple assembly of FIG. 1A in a deployed position with tissue not depicted for the purpose of clarity.

FIG. 3 is a perspective view of a fastening device attached to the distal end of an endoscope in a non-deployed position according to an embodiment of the present disclosure.

FIG. 4 is a perspective view of the fastening device of FIG. 3 in a deployed position with tissue not depicted for the purpose of clarity.

FIG. 5 is an exploded view of the fastening device of FIG. 3.

FIG. 6 is a bottom view of another embodiment of a fastening device in a stowed position according to an embodiment of the present disclosure with the casing exposed for the purpose of clarity.

FIG. 7 is a bottom view of the fastening device of FIG. 6 in a non-stowed position.

FIG. 8 is a front view of the fastening device of FIG. 6 attached to the distal portion of an endoscope.

FIG. 9 is a side view of the fastening device and endoscope illustrated in FIG. 8.

FIG. 10 is a perspective view of the fastening device of FIG. 6 in a non-deployed position attached to an endoscope and having forceps extending through a channel of the endoscope.

FIG. 11 is a perspective view of the fastening device of FIG. 10 in a deployed position.

FIG. 12 is a schematic side view of a rivet assembly in a non-deployed position according to an embodiment of the present disclosure.

FIG. 13 is a schematic side view of the rivet assembly of FIG. 12 in a deployed position.

FIG. 14 is a schematic side view of another embodiment of a rivet assembly in a non-deployed position according to an embodiment of the present disclosure.

FIG. 15 is a schematic side view of the rivet assembly of FIG. 14 in a deployed position.

FIG. 16 is a perspective view of a fastening device in a non-deployed position (with tissue not depicted for the purpose of clarity) attached to an endoscope according to an embodiment of the present disclosure.

FIG. 17 is a perspective view of the fastening device of FIG. 16 in a deployed position with tissue not depicted for the purpose of clarity.

FIG. 18 is an exploded view of the fastening device of FIG. 16.

### DETAILED DESCRIPTION

The present disclosure generally relates to surgical staple assemblies, surgical rivet assemblies, surgical fastening devices, surgical stapling systems and surgical fastening kits. Such devices are configured for placement in a patient's body and are therefore sterile and constructed of biocompatible materials. The present disclosure refers to the term “substantially” with respect to certain orientations or positions. By “substantially” is meant that the orientation or position of the element need not have the mathematically exact described orientation or position but can have an orientation or position that is recognizable by one skilled in the art as generally or approximately having the described orientation or position. The disclosure also refers to the term “top,” “bottom,” “left” and “right,” with respect to a staple. These terms refer to portions of the staple or when the staple is in a non-deployed, upright position as illustrated in FIG. 1. Further, with respect to a staple, staple assembly or rivet assembly, the term “non-deployed” or “pre-deployed” refers to the position of the staples, staple assemblies or rivet assemblies when they have not been deployed in the body and coupled together to fixate tissue between the staples, staple assemblies or rivet assemblies. The term “deployed” refers to the position of the fastened tissue when the staples, staple assemblies or rivet assemblies have been deployed in the body and coupled together to fixate tissue between the staples, staple assemblies or rivet assemblies. With respect to a fastening device or fastening system, the term “non-deployed” refers to the position of the fastening device or fastening system before a staple, staple assembly or rivet assembly has been deployed in the body to fixate tissue. The term “deployed” refers to the position of the fastening device or fastening system when a staple, staple assembly or rivet assembly has been deployed into the body and has fastened tissue together.

As stated above, in certain embodiments, the present disclosure provides surgical staples that can be used to repair tissue in a patient's body. FIG. 1 depicts a duplex staple assembly 10 comprising a first staple 12 and a second staple 32. First staple 12 comprises a base 14 having a back surface 16, a front surface 18, a top portion 20, a bottom portion 22, a left side portion 24 and a right side portion 26. First staple 12 also has a left prong 28 extending from left side portion 24 of base 14 and a right prong 30 extending from right side portion 26 of base 14. Second staple 32 comprises a base 34 having a back surface 36, a front surface 38, a top portion 40, a bottom portion 42, a left side portion 44 and a right side portion 46. Second staple 32 also has a top prong 48 extending from top portion 40 of base 34 and a bottom prong 50 extending from bottom portion 42. FIG. 1 illustrates the duplex staple assembly in a non-deployed or pre-deployed position. Referring to FIG. 2, in a deployed position (tissue between staples not shown for purposes of clarity), left prong 28 and right prong 30 of first staple 12 are retroflexed onto back surface 36 of second staple 32 and top prong 48 and bottom prong 50 (not shown) of second staple 32 are retroflexed onto back surface 16 of first staple 12. Referring to FIG. 5, the face of plunger 68 of an exemplary fastening device 60 is in the shape of a cross. This pattern is a curved indentation that performs the action of plastically deforming the staple prongs from the opposing staple, bending the prongs back (retroflexing) on the back of the opposite staple after the prongs have passed through the tissue.

In certain embodiments, top portion 20, bottom portion 22, left side portion 24, and right side portion 26 of first staple 12 each define a substantially central indentation (21, 23, 25, and 27 respectively) as shown in FIG. 1. Left prong 28 extends from substantially central indentation 25 of left side portion 24 and right prong 30 extends from substantially central indentation 27 of right side portion 26 of first staple 12. Top portion 40, bottom portion 42, left side portion 44 and right side portion 46 of second staple 32 also each define a substantially central indentation (41, 43, 45, and 47). Top prong 48 extends from substantially central indention 41 of top portion 40 and bottom prong 50 extends from substantially central indentation 43 of bottom portion 42 of second staple 32. As shown in FIG. 2, left prong 28 and right prong 30 of first staple 12 are disposed in the respective substantially central indentation 45 and 47 of left side portion 44 and right side portion 46 of second staple 32 and are retroflexed onto the back surface of the second staple in a deployed position. Top prong 48 and bottom prong 50 of second staple 32 are disposed in the respective substantially central indentation 21 and 23 of top portion 20 and bottom portion 22 of first staple 12 and are retroflexed onto the back surface of first staple 12 in a deployed position. Although only two prongs are illustrated for each of the first and second staples, the staples can each have more than two prongs.

Referring to FIG. 5, the face of plunger 68 of an exemplary fastening device 60 is in the shape of a cross. This pattern is a curved indentation that performs the action of plastically deforming the staple prongs from the opposing staple, bending the prongs back (retroflexing) on the back of the opposite staple after the prongs have passed through the tissue.

A duplex staple assembly can provide a redundant stapling approach that differs from existing staples. The staples can have a large tissue contact surface that allows for the stapling load to be distributed over the surface of the approximated tissue to be repaired. For example, the front surface of the staples, which is the surface that contacts tissue, can have a surface area of between approximately 9 mm² and approximately 36 mm². The front surfaces of the staples can have larger surface areas to accommodate larger portions of the anatomy, such as the large intestine.

Referring to FIGS. 1A and 2A, a staple assembly 700 can comprise a staple 702 and a staple back 704. Staple 702 comprises a base 714 having a back surface 716, a front surface 718, a top portion 720, a bottom portion 722, a left side portion 724 and a right side portion 726. First staple 702 also has a left prong 728 extending from left side portion 724 of base 14, a right prong 730 extending from right side portion 726 of base 714, a top prong 732 extending from top portion 720 of base 714 and a bottom prong 734 extending from bottom portion 722. Staple back 704 comprises a base 714 having a back surface 735, a front surface 738, a top portion 740, a bottom portion 742, a left side portion 744 and a right side portion 746. FIG. 1A illustrates the staple assembly 700 in a non-deployed or pre-deployed position. Referring to FIG. 2A, in a deployed position (tissue between staple and staple back not shown for purposes of clarity), left prong 728, right prong 730, top prong 732, and bottom prong 734 of staple 702 are retroflexed onto back surface 735 of staple back 704.

In either embodiment of a staple assembly illustrated in FIG. 1, 2, 1A or 2A, the prongs of one staple are retroflexed onto the back of the opposing staple or staple back thereby preventing excessive damage to the stapled tissue caused by the endpoints of the opposing staple prongs or one of the staples. Such qualities are beneficial when used in patients with excessively inflamed and friable tissue, preventing the staple from ripping through the tissue due to causes such as GI peristaltic activity or increases in intraluminal pressure and limiting the recurrences of perforations.

Certain embodiments of the present disclosure provide a fastening device that can be used to load and discharge surgical staples. Such surgical staples can be surgical duplex staple assemblies as described herein, other surgical staples or rivet assemblies as disclosed herein. Referring to FIG. 3, in an embodiment, a fastening device 60 comprises a casing 62 that is sized and configured to house a plurality of staples as disclosed herein, a plurality of rivet assemblies as disclosed herein, or other surgical staples and fasteners. Preferably, casing 62 or another suitable casing of a surgical fastening device has a length of between approximately 20 mm and approximately 36 mm, a width of between approximately 10 mm and approximately 15 mm and a height of between approximately 5 mm approximately 7 mm. Fastening device 60 further includes a first carrier 64 and second carrier 66 opposite first carrier 64, both of which are disposed within casing 62. First carrier comprises a first plunger 68 disposed therein and second carrier 66 comprises a second plunger 70 disposed therein. As depicted in FIG. 5, a plurality of first staples 12 can be sequentially disposed in first carrier 64 and a plurality of second staples 32 can be sequentially disposed in second carrier 66. A carrier clamp can be actuated using a steel cable system that is operated outside the body by an endoscopist. The operation controls can be a hand operated cable system that connects to the fastening device actuator by running through a small diameter tube external to the shaft of the endoscope, but secured safely to the shaft using, for example, small clip-on spring loaded band-style clamps affixed at multiple locations along the endoscope's length. The arrows depicted in FIG. 3 indicate the direction in which the carriers are urged together to deploy a staple, rivet assembly or other fastener.

FIG. 5 is an exploded view of exemplary components of fastening device 60 illustrated in FIG. 3. Casing 62 comprises a base 72 attached to a cover 74 via fasteners 76 and a rear casing 73 attached to both base 72 and cover 74. First carrier 64 and second carrier 66 each define a plurality of openings sized and configured to accept guide rails 71 which ensure that carriers 64 and 66 are aligned in casing 62. First carrier 64 also comprises a first staple plunger 68 disposed therein and second carrier 66 comprises a second staple plunger 70 disposed therein. Also housed within first carrier 64 is a first staple loader 78 and a first staple plunger tension spring 80. Similarly, housed within second carrier 66 is a second staple loader 82 and a second staple plunger tension spring 84. The plurality of first staples 12 are retained by first staple retainer 86 and the plurality of second staples 32 are retained by second staple retainer 88. First and second staple retainers 86 and 88 are coupled to respective first and second carrier covers 90 and 92 respectively which, in turn, are attached to respective first and second carriers 64 and 66. Portions of first and second staple retainers 86 and 88 are disposed within a staple retainer guide rail 98 to ensure staple retainers 86 and 88 are aligned in casing 62 during and after staple deployment. Positioned between first and second carrier covers 90 and 92 are carrier compression springs 94 and 96, which allow carriers 64 and 66 to return to a non-deployed configuration after a staple has been discharged from the carriers. The aforementioned components of a surgical fastening device are only exemplary and components can be replaced with different components or excluded all together so long as the fastening device has a low profile and can be attached to the bottom portion of the distal end of a flexible endoscope so as to not occlude the operator's field of view as described in more detail below. Further, the above described components can be used to deploy rivet assemblies as disclosed herein and other fasteners.

In further detail, the cover fasteners secure the endoscopic fastening device cover to the endoscopic fastening device base. The endoscopic fastening device cover protects the inner components of the fastening device and provides support for the guide rails. The guide rails guide the translational pinching motion of the carriers during the stapling action. The staple plunger deploys the staple into the approximated tissue and deforms the prongs of the opposing staple using the curved indentation on the face of the plunger so that the staple can be permanently fixated in the approximated tissue. The staple plunger tension spring maintains a tension force on the staple loader so that the staple in the carrier can be loaded into the distal end of the carrier just prior to being deployed into the tissue. The staple loader slides along the interior of the carrier progressing the staples in the direction of the distal end of the carrier as the staples are deployed. The carrier serves multiple functions such as acting as a reservoir for the staples, securing the approximated tissue for staple deployment with the pinching action, and acting as the primary support for the staple plunger. A duplex staple assembly is deployed into approximated tissue to fixate opposing tissue together during perforation repair. The staple retainer secures staples in place to prevent the loaded staple from falling out of the open end of the carrier while also keeping the staples in-line and preventing them from jamming the fastening device due to staple overlap. The proximal end of the staple retainer is connected to the spring-loaded staple retainer guide rail. The carrier cover closes the interior of the carrier preventing the staples and staple retainer from falling out of the carrier while also serving as a guide for the translation retracting motion of the staple retainer. The carrier compression spring opposes the carrier cover and ensures that the carrier is resting in the open position. The staple retainer compression spring 99 opposes the staple retainer guide rail and ensures that the fastening device retainer is fully extended when not in use to prevent staples from falling out of the distal end of the carrier. The staple retainer cable pin 33 inserts through a center hole of the staple retainer guide rail and the rear casing where it is attached to a cable that is controlled by the operator. It allows for the actuation of the fastening device retainer guide rail that retracts the staple retainer for staple deployment. The staple retainer guide rail retracts the staple retainer for staple deployment while also serving a guide rail for the pinching motion of the carriers. The rear casing covers the rear portion of the endoscopic fastening device while also providing a normal force for the staple retainer compression spring. The carrier slide-crank cap 75 secures the top carrier slide-crank 77 and the bottom carrier slide-crank 79 to the casing base while also serving as the pivot point of both slide cranks. The top carrier slide-crank serves as the cable driven actuation motion that causes the carrier to have a pinching motion by converting a translational motion of the cable (operated by the endoscopist) into a rotational motion about the slide-crank cap. The cable is connected to a small hole on the proximal end of the top carrier slide-crank. A slotted hole on the distal end of the top carrier slide-crank attaches to a cylindrical peg (not shown) on the bottom side of the carrier allowing the conversion of the rotational scissor like motion of the carrier slide-crank into a translational motion that has a force vector 90 degrees from the cable force vector during staple deployment. The casing base serves as the base for all interior components within the casing. A carrier fastener 81 inserts through a hole in the bottom of the casing base and screws into the bottom of the carrier slide-crank cap.

As shown in FIG. 3, fastening device 60 has a low profile and can be attached to the bottom portion 102 of the distal end 104 of a flexible endoscope 100. Endoscope 100 can define a plurality of channels 106 that extend through the lumen of endoscope 100. Such channels can accommodate different types of surgical instrumentation such as suction or biopsy devices or can be used to deliver air or water to the surgical site or surgical pathway. As shown in FIG. 3, channel 106a has a lens 108 attached to the distal end thereof to allow the operator to view the surgical pathway or the surgical site. Because fastening device 60 is low profile, it can be attached to bottom portion 102 of distal end 104 of endoscope 100, as shown in FIG. 3, without occluding or obstructing channels 106 and the operator's field of view. FIG. 3 illustrates fastening device 60 in an open or non-deployed configuration and FIG. 4 illustrates fastening device 60 in a closed or deployed configuration. The fastening device can either be manually attached to the distal end of an endoscope by the operator or pre-attached to an endoscope during manufacturing.

FIGS. 6 and 7 depict another embodiment of a fastening device that can be used to deploy staples as disclosed herein, rivet assemblies as disclosed herein other surgical staples or surgical fasteners. Fastening device 200 comprises a clamp 202 that has opposing first and second arms 204 and 206. Each arm has an exit port 208 and 210 at a respective distal end 212 and 214 thereof. Fastening device 200 also includes a spring-loaded casing 216 in operable association with clamp 202 and located between first and second arms 204 and 206 of clamp 202 in a stowed position (FIG. 6). The cover of the casing is not shown for the purpose of clarity in FIGS. 6 and 7. Spring-loaded casing 216 has a first section 218 and a second section 220. First section 218 has a distal access port 222 facing first arm 204 and second section 206 also has a distal access port 224 facing second arm 206. First section 218 is sized and configured to house a plurality of first staples or rivets of rivet assemblies and second section 220 is sized and configured to house a plurality of complementary second staples or secondary heads of rivet assemblies. Fastening device 200 further includes a rod 226 in slidable relation with clamp 202 and located distal to spring-loaded casing 216.

In the stowed configuration illustrated in FIG. 6, first and second arms 204 and 206 of clamp 202 are positioned over respective access ports 222 and 224 to allow staples 230 to be loaded into the respective distal ends 212 and 214 of first and second arms 204 and 206. Once the staples are loaded into clamp 202, as depicted in FIG. 7, springs 236 and 238 can advance the next pair of staples into position for loading onto clamp 202. The loaded clamp can be advanced along the sliding direction (indicated by the arrow in FIG. 7) towards the perforation site in the patient's body so that the fastening device may install the staples into the tissue. Although FIGS. 6 and 7 depict surgical staplers, rivet assemblies and other fasteners could be used as well.

As with other fastening devices described herein, fastening device 200 preferably has a low profile configuration and can be attached to the distal end of an endoscope 228 such as a flexible endoscope as illustrated in FIGS. 8 and 9. The fastening device can either be manually attached to the distal end of an endoscope by the operator or pre-attached to an endoscope during manufacturing. As can be seen from FIGS. 8 and 9, in a stowed position, fastening device 200 maintains a low profile so that the outer diameter preferably does not exceed the nominal inner diameter of the lower esophageal spincter (approximately 13 mm), allowing easy passage from the esophagus to the stomach.

Surgical fastening devices as described herein can be used for various surgical purposes, including to close gastrointestinal perforations. For example, FIGS. 10 and 11 depict fastening device 200 attached to the distal end of an endoscope 240. Once delivered to the perforation site 242 in the gastric wall 244 of a patient's body, the tissue is approximated using endoscopic forceps 246. Fastening device 200 can then staple or fixate the approximated tissue. Fastening device 200 can be equipped with a reservoir of staples 230 or rivet assemblies to allow the repair to be completed without removing fastening device 200 to reload it.

The present disclosure also provides surgical fastening systems that include the duplex staple assembly disclosed herein and a surgical fastening device to deploy the duplex staple assembly. Such fastening systems can also include an endoscope to which the surgical fastening device is attached. Other embodiments provide a surgical fastening system that includes a surgical fastening device as described herein and staples, rivet assemblies as disclosed herein, or other fastneres, that can be deployed by the surgical fastening device. Such surgical stapling systems can also include an endoscope to which the surgical fastening device is attached.

Referring to FIG. 12 and FIG. 13, certain embodiments provide a rivet assembly 300 comprising a rivet 302 and a secondary head 304. Rivet 302 comprises a primary head 306, a shaft 308 extending from the primary head 306 and an expandable tail 310 extending from the shaft 308. Secondary head 304 comprises a secondary head body 312 defining a substantially central hole 314 that is sized and configured to receive tail 310 of rivet 302 in a deployed configuration. As seen in FIGS. 12 and 13, during tissue repair, rivet 302 is deployed in a manner so that it penetrates the approximated tissue 316 and tail 310 inserts into substantially central hole 314 of secondary head 304. Rivet assembly 300 can be used with a deployment device that expands the tail 310 of rivet 302, which prevents disengagement of secondary head 304 from rivet 302.

After deployment of rivet assembly 300, the approximated tissue 316 is compressed and secured between the primary head 306 of rivet 302 and secondary head 304 as seen in FIG. 13. Such rivet assemblies can be deployed along the perforation line of the tissue until the tissue defect is repaired. The rivets can be part of a kit including rivets having different lengths to allow for repair of tissue varying in thicknesses.

Referring to FIGS. 14 and 15, in certain embodiments, a rivet assembly 400 comprises a rivet 402 and a secondary head 404. Rivet 402 comprises a primary head 406, a shaft 408 extending from primary head 406, and a ridged tail 410 having one or more outer ridges extending from ridged shaft 408. In certain embodiments, shaft 408 has one or more outer ridges as well. Secondary head 404 comprises a secondary head body 412 defining a substantially central hole 416 that is sized and configured to receive tail 410 of rivet 402 and internal locking tabs 418 radially extending into substantially central hole 416. Internal locking tabs 418 secure ridged ridged tail 410 and also can secure ridged shaft 408 in place as rivet 402 is advanced during the insertion process. During tissue repair, rivet 400 is deployed in a manner so that ridged shaft 408 and ridged tail 410 penetrate the approximated tissue 420 and insert into the substantially central hole 416 of secondary head 404. Ridged tail 410 passes through the secondary head body 412 of secondary head 404. As ridged shaft 408 and ridged tail 410 of rivet 402 advance through tissue, the internal tabs 418 of secondary head 404 prevent rivet 402 from backing out and the compression pressure increases. Rivet insertion depth determines the final tissue approximation distance compression force, securing the tissue between the primary head of the rivet and the secondary head. Such ridged rivets can be deployed along the perforation line of tissue until the tissue defect is repaired in the same manner as other rivets described herein.

Rivets and staples as described herein can be fabricated from dissolvable material.

Referring to FIG. 16-18, a surgical fastening kit is provided. Such a kit includes a plurality of rivet cartridges 502. Each rivet cartridge 502 comprises a plurality of rivets 504. Each rivet 504 comprises a primary head 506, a shaft 508 extending from primary head 506, and a tail 510 extending from shaft 508. The surgical kit also includes a plurality of secondary head cartridges 512. Each secondary head cartridge 512 comprises a plurality of secondary heads 514. Each secondary head 514 defines an opening sized to receive the tail 510 of one of the rivets 504. In addition or alternatively, the kit includes a plurality of staple cartridges 516 and a plurality of staple back cartridges 518. Each staple cartridge 516 comprises a plurality of staples 520 and each staple back cartridge 518 comprises a plurality of staple backs 524. The kit also includes a tension-loaded fastening device 526 comprising a first arm 528 defining a compartment 530 sized to receive one of the plurality of rivet cartridges 502 or one of the plurality of staple cartridges 516. The tension-loaded fastening device 526 also includes a second arm 532 opposing first arm 528 and that defines a compartment 534 sized to receive one of the plurality of secondary head cartridges 512 or one of the plurality of staple back cartridges 518. The tension-loaded fastener device 526 is configured to attach or is pre-attached to a bottom portion of a distal end of a flexible endoscope defining a plurality of channels such that the tension-loaded fastener device does not occlude a field of view of a camera, a light source or the plurality of channels of the endoscope as shown in FIGS. 16 and 17.

In certain embodiments as disclosed above, the rivet tail comprises an outer ridge and each of the secondary heads comprises an internal tab that engages the outer ridge of the tail in a deployed configuration. In certain embodiments, the rivet shaft also comprises one or more outer ridges.

In more detail with reference to FIG. 18, rivet cartridge 502 holds a plurality of rivets and deploys a single rivet through an opening 540 at a distal portion of rivet cartridge 502. Secondary head cartridge 512 holds a plurality of secondary heads and deploys a single secondary head with each deployment. Rivet shaft 508 and rivet tail 510 of rivet 504 penetrates through approximated tissue as illustrated in FIGS. 14 and 15. Secondary head 514 securely holds a rivet 504 to allow the approximated tissue to be held tightly together as illustrated in FIG. 15. Staple cartridge 516 holds a plurality of staples and deploys a single staple through the substantially square-shaped opening 542 at a distal portion of staple cartridge 516. Staple back cartridge 518 holds a plurality of staple backs and deploys a single staple back with each deployment. Staple 520 penetrates through approximated tissue and staple back 524 securely holds staple 52 to allow the approximated tissue to be held tightly together. An advancer 536 advances the reservoir of staples, staple backs, rivets, or secondary heads within the respective cartridges so that these fasteners are in the proper position as they are deployed into tissue. An elastic band 538 provides actuation force for each advancer 536. A plunger 544 is actuated to deploy a rivet 504 or staple 520 into tissue and a plunger retraction spring 546 retracts plunger 544 after each deployments. First arm 528 of tension-loaded fastening device 526 holds the rivet cartridge 502 and, optionally, the staple cartridge 516. Second arm 532 holds the secondary head cartridge 512 and, optionally, the staple back cartridge 518.

The kit also includes a tension-loaded fastening device 526 comprising a first arm 528 defining a compartment 530 sized to receive one of the plurality of rivet cartridges 502 or one of the plurality of staple cartridges 516. The tension-loaded fastening device 526 also includes a second arm 532 opposing first arm 528 and that defines a compartment 534 sized to receive one of the plurality of secondary head cartridges 512 or one of the plurality of staple back cartridges 518. An endoscope interface bracket 542 couples to a bottom portion of a distal end of a flexible endoscope defining a plurality of channels such that the tension-loaded fastener device does not occlude a field of view of a camera, a light source or the plurality of channels of the endoscope as illustrated in FIGS. 16 and 17.

As such, tension-loaded fastening device 526 can attach to the distal end of a flexible endoscope 600. Controlled by an endoscopist, the fastening device deploys staple components or rivet components from respective cartridges into the approximated tissue. The cartridges store a plurality of staple components or rivet components in the reservoir of each cartridge with one staple component or rivet component loaded in the fastening device's deployment chamber. Once emptied, the cartridges can be removed and replaced with cartridges loaded with staple components and rivet components.

The use of surgical fastening devices as described herein may give rise to less post-operative complications associated with percutaneous incisions, such as shorter operating times, reduced infection and reduced cosmetic patient concerns. Using devices as disclosed herein with a flexible endoscope allows the devices to perform surgical repairs on the lower esophagus, gastric wall, small intestine, large intestine, and rectum due to the tortuous anatomy of the upper and lower GI tracts. In particular, surgical stapling devices can be used to perform a natural orifice transluminal endoscopic surgery procedure by allowing an operator to access the peritoneal cavity from the GI tract to perform organ resections of the liver, pancreas, spleen and kidney. Other uses include endoscopic tumor biopsy and resection of tumors located in the peritoneal or retroperitoneal space, lymph node biopsy for cancer staging, visual inspection of the peritoneal cavity in place of or complementing open or laparoscopic exploratory surgery, repair of anastomotic leaks which is common following bariatric surgery, cholesystectomy, endoscopic hernia repair, Salpingo-oofrectomy, adhesiolysis, resection of tumor on the vertebrae, removal of endometriosis, and removal of a heterotopic rest.

Each of the disclosed aspects and embodiments of the present disclosure may be considered individually or in combination with other aspects and embodiments. Further, while certain features of embodiments may be shown in only certain figures, such features can be incorporated into other embodiments shown in other figures or otherwise disclosed in the specification. Additionally, when describing a range, all points within that range are included in this disclosure. In addition, unless otherwise specified, none of the steps of the methods of the present invention are confined to any particular order of performance.

What is claimed is:

1. A surgical duplex staple assembly comprising:
a first staple comprising:
a base having a back surface and a front surface and having a top portion,
a bottom portion, a left side portion, and a right side portion;
a left prong extending from the left side portion; and
a right prong extending from the right side portion;
a second staple comprising:
a base having a back surface, a front surface, a top portion, a bottom portion, a left side portion, and a right side portion;
wherein in a deployed position, the left and right prong of the first staple penetrate through tissue and are retroflexed onto the back surface of the second staple.

2. The surgical duplex staple assembly of claim 1, wherein the first staple further comprises a top prong extending from the top portion of the base and a bottom prong extending from the bottom portion of the base, wherein in a deployed position, the top and bottom prong of the first staple penetrate through tissue and are retroflexed on the back surface of the second staple.

3. The surgical duplex staple assembly of claim 1 wherein the second staple comprises a top prong extending from the top portion of the base and a bottom prong extending from the bottom portion of the base, wherein in a deployed position, the left and right prong of the first staple penetrate through tissue and are retroflexed onto the back surface of the second staple, and the top and bottom prong of the second staple penetrate through tissue and are retroflexed onto the back surface of the first staple.

4. The surgical duplex staple assembly of claim 3, wherein:
the top portion, the bottom portion, the left side portion, and the right side portion of the first and the second staple each define a substantially central indentation;
the left prong and the right prong of the first staple extend from the respective substantially central indentation of the left side portion and the right side portion of the first staple;
the top prong and the bottom prong of the second staple extend from the respective substantially central indentation of the top portion and the bottom portion of the second staple;
the right prong and the left prong of the first staple are disposed in the respective substantially central indentation of the right side portion and the left side portion of the second staple and are retroflexed onto the back surface of the second staple in a deployed configuration; and
the top prong and the bottom prong of the second staple are disposed in the respective substantially central indentation of the top portion and the bottom portion of the first staple and are retroflexed onto the back surface of the first staple in a deployed configuration.

5. The surgical duplex staple assembly of claim 1, wherein the front surface of the first and second staple each have a surface area of between approximately 9 mm² and approximately 36 mm².

6. A surgical fastening system comprising:
the surgical duplex staple assembly of claim 1; and
a fastening device configured to house the surgical duplex staple assembly.

7. The surgical fastening system of claim 6, further comprising a flexible endoscope defining a plurality of channels, the fastening device being configured to attach or is pre-attached to a bottom portion of a distal end of the flexible endoscope such that the fastening device does not occlude a field of view of a camera, a light source or the plurality of channels of the endoscope.

8. A fastening device comprising:
a casing;
a first carrier disposed within the casing and having a distal end defining an exit opening, the first carrier configured to house a plurality of the first staples of claim 1;
a second carrier disposed within the casing opposite the first carrier and having a distal end defining an exit opening, the second carrier configured to house a plurality of
the second staples of claim 1 therein;
a first plunger disposed within the first carrier;
a second plunger disposed within the second carrier; and
a carrier compression spring connected to the first and second carriers.

9. The fastening device of claim 8, wherein the casing comprises a base that is configured to attach or is pre-attached to a bottom portion of a distal end of a flexible endoscope defining a plurality of channels, the base attachable or pre-attached to the flexible endoscope such that the casing does not occlude a field of view of a camera, a light source, or the plurality of channels of the endoscope.

10. A surgical fastening device comprising:
a casing;
a first carrier disposed within the casing and having a distal end defining an exit opening, the first carrier configured to house a plurality of first staples or a plurality of rivets of rivet assemblies;
a second carrier disposed within the casing opposite the first carrier and having a distal end defining an exit opening, the second carrier configured to house a plurality of complementary second staples or a plurality of secondary heads of rivet assemblies therein;
a first plunger disposed within the first carrier;
a second plunger disposed within the second carrier; and
a carrier compression spring connected to the first and second carriers, wherein the casing comprises a base that is configured to attach or is pre-attached to a bottom portion of a distal end of a flexible endoscope defining a plurality of channels, the base attachable or pre-attached to the flexible endoscope such that the casing does not occlude a field of view of a camera, a light source or the plurality of channels of the endoscope.

11. A fastening device comprising:
a clamp comprising opposing first and second arms, each arm having an exit port at a distal end thereof;
a spring-loaded casing in operable association with the clamp and located between the first and second arms of the clamp in a stowed position, the spring-loaded casing comprising:
a first section having a distal access port facing the first arm, the first section configured to house a plurality of the first staples of claim 1 therein;
a second section having a distal access port facing the second arm, the second section configured to house a plurality of the second staples of claim 1 therein;
and
a rod in slidable relation with the clamp and located distal to the spring-loaded casing.

12. The fastening device of claim 11, wherein the clamp and spring-loaded casing are configured to attach or are pre-attached to a bottom portion of a distal end of a flexible endoscope defining a plurality of channels such that the fastening device does not occlude a field of view of a camera, a light source or the plurality of channels of the endoscope.

13. A surgical fastening device comprising:
a clamp comprising opposing first and second arms, each arm having an exit port at a distal end thereof;
a spring-loaded casing in operable association with the clamp and located between the first and second arms of the clamp in a stowed position, the spring-loaded casing comprising:
a first section having a distal access port facing the first arm, the first section configured to house a plurality of first staples or a plurality of rivets of rivet assemblies therein;
a second section having a distal access port facing the second arm, the second section configured to house a plurality of second complementary staples or a plurality of secondary heads of rivet assemblies therein; and
a rod in slidable relation with the clamp and located distal to the spring-loaded casing, wherein the clamp and spring-loaded casing are configured to attach or are pre-attached to a bottom portion of a distal end of a flexible endoscope defining a plurality of channels such that the fastening device does not occlude a field of view of a camera, a light source or the plurality of channels of the endoscope.

14. The surgical fastening system of claim 13, further comprising forceps extending from the distal end of the flexible endoscope.

15. A surgical fastening kit comprising:
a plurality of rivet cartridges, each rivet cartridge comprising a plurality of rivets, each rivet comprising a primary head, a shaft extending from the primary head, and a tail extending from the shaft;
a plurality of secondary head cartridges, each secondary head cartridge comprising a plurality of secondary heads, each secondary head defining an opening sized to receive the tail of one of the rivets;
a plurality of staple cartridges, each staple cartridge comprising a plurality of staples;
a plurality of staple back cartridges, each stage back cartridge comprising a plurality of staple backs, each staple back configured to couple to a staple;
a tension-loaded fastening device comprising:
a first arm defining a compartment sized to receive one of the plurality of rivet cartridges or one of the plurality of staple cartridges;
a second arm opposing the first arm, the second arm defining a compartment sized to receive one of the plurality of secondary head cartridges or one of the plurality of staple back cartridges, wherein the tension-loaded fastener device is configured to attach or is pre-attached to a bottom portion of a distal end of a flexible endoscope defining a plurality of channels such that the tension-loaded fastener device does not occlude a field of view of a camera, a light source or the plurality of channels of the endoscope.

16. The fastening kit of claim 15, wherein the tail of each of the rivets comprises an outer ridge and each of the secondary heads comprises an internal tab that engages the outer ridge of the tail.

17. The fastening kit of claim 15, wherein the shaft of each of the rivets comprises an outer ridge.
